(19) United States
(12) Patent Application Publication
(10) Pub. No.: US 2025/0303119 A1
(43) Pub. Date: Oct. 2, 2025

(54) ACCESS DEVICE WITH SENSORS

(71) Applicant: Boston Scientific Scimed, Inc., Maple Grove, MN (US)

(72) Inventors: Ali Ziegler, Waltham, MA (US); Cassandra Anne Hailson, Marlborough, MA (US); Kenneth Flynn, Woburn, MA (US); Jeffrey A. Meganck, N Grafton, MA (US); Patrick N. Leugue, Shrewsbury, MA (US); Niraj Prasad Rauniyar, Plymouth, MN (US)

(21) Appl. No.: 19/088,038

(22) Filed: Mar. 24, 2025

Related U.S. Application Data
(60) Provisional application No. 63/569,810, filed on Mar. 26, 2024.

(51) Int. Cl. A61M 25/06; A61M 25/00

(52) U.S. Cl. CPC A61M 25/0662; A61M 2025/0002; A61M 2205/3334

(57) ABSTRACT

According to a general aspect, an apparatus having an elongate member defining a lumen. The elongate member being configured to be disposed within a body of a patient. The apparatus having a flow meter configured to measure fluid flow through the lumen of the elongate member. The apparatus also having a pressure sensor.

### CROSS-REFERENCE TO RELATED APPLICATION

This application claims priority to U.S. Provisional Patent Application No. 63/569,810, filed on Mar. 26, 2024, entitled “ACCESS DEVICE WITH SENSORS”, the disclosure of which is incorporated by reference herein in its entirety.

### TECHNICAL FIELD

This disclosure relates generally to bodily access devices and, more specifically, to bodily access devices that include sensors.

### BACKGROUND

Access devices may be used to gain access to various locations within a body of a patient. For example, tubular members or sheaths may be inserted into a body of a patient to gain access to a specific location within the body of the patient. In some cases, access to such locations within the body of the patient may be used to perform further medical procedures. In some examples, a urological access device may be used to access a kidney of a patient. Such access may be used to perform additional medical procedures such as lithotripsy procedures, percutaneous nephrolithotomy procedures, and urodynamics.

### SUMMARY

According to a general aspect, an apparatus having an elongate member defining a lumen. The elongate member being configured to be disposed within a body of a patient. The apparatus having a flow meter configured to measure fluid flow through the lumen of the elongate member. The apparatus also having a pressure sensor.

In some implementations, the flow meter is disposed within the lumen defined by the elongate member. In some implementations, the pressure sensor is disposed outside of the lumen defined by the elongate member.

In some implementations, the elongate member includes a sidewall that has an inner surface that defines the lumen and an outer surface opposite the inner surface, the flow meter is disposed adjacent the inner surface of the sidewall. In some implementations, the elongate member includes a sidewall that has an inner surface that defines the lumen and an outer surface opposite the inner surface, the pressure sensor is disposed adjacent the outer surface of the sidewall.

In some implementations, the lumen defined by the elongate member is a first lumen, the elongate member defining a second lumen. In some implementations, the lumen defined by the elongate member is a first lumen, the elongate member defining a second lumen and a third lumen. In some implementations, the lumen defined by the elongate member is a first lumen, the elongate member defining a second lumen and a third lumen, the first lumen being configured to receive a secondary medical device, the first lumen configured to be an inflow channel, the second lumen configured to be an outflow channel. In some implementations, the lumen defined by the elongate member is a first lumen, the flow meter being a first flow meter, the elongate member defining a second lumen, the apparatus further comprising a second flow meter disposed within the second lumen. In some implementations, the lumen defined by the elongate member is a first lumen, the flow meter being a first flow meter, the elongate member defining a second lumen and a third lumen, the apparatus further comprising a second flow meter disposed within the second lumen.

In some implementations, the apparatus further includes a wire operatively coupled to the flow meter, at least a portion of the wire being disposed within lumen defined by the elongate member.

In some implementations, the apparatus of further includes a wire operatively coupled to the flow meter, at least a portion of the wire being disposed within a sidewall of the elongate member.

In some implementations, the apparatus further includes a display member coupled to a proximal end portion of the elongate member.

In some implementations, the apparatus further includes a display member configured to be disposed a distance from the elongate member and configured to receive information regarding the fluid flow within the lumen of the elongate member.

In some implementations, the apparatus further includes a display member configured to be disposed a distance from the elongate member and configured to wirelessly communicate with the elongate member.

According to a general aspect, an apparatus includes an elongate member defining a first lumen, a second lumen and a third lumen, the elongate member being configured to be disposed within a body of a patient, the first lumen being configured to receive a secondary medical device; a first flow meter configured to measure fluid flow through the second lumen of the elongate member; and a second flow meter configured to measure fluid flow through the third lumen of the elongate member.

In some implementations, the apparatus includes a pressure sensor. In some implementations, the apparatus includes a pressure sensor coupled to an end portion of the elongate member. In some implementations, the apparatus includes a display member coupled to the elongate member.

In some implementations, the apparatus includes a display member configured to be disposed a distance from the elongate member and configured to wirelessly communicate with the elongate member.

### BRIEF DESCRIPTION OF THE DRAWINGS

FIG. 1 schematically illustrates an apparatus according to an implementation.

FIG. 2 schematically illustrates an apparatus according to an implementation.

FIG. 3 is a perspective view of an apparatus according to an implementation.

FIG. 4 is a cross-sectional view of the apparatus of FIG. 3.

FIGS. 5 and 6 are cross-sectional views of the apparatus of FIG. 3.

FIG. 7 is a cross-sectional view of an apparatus according to an implementation.

FIG. 8 is a perspective view of an apparatus according to an implementation.

FIG. 9 is a cross-sectional view of the apparatus of FIG. 8.

FIGS. 10 and 11 are cross-sectional views of an apparatus of FIG. 8.

FIG. 12 is a cross-sectional view of an apparatus according to an implementation.

FIGS. 13 and 14 are perspective views of devices or systems according to implementations.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as access devices or access sheaths that may be placed within a body of a patient to allow for or provide access to an internal portion of the body of the patient. The term patient may hereafter be used for a person who benefits from the medical device or the methods disclosed in the present disclosure. For example, the patient can be a person whose body receives the medical device or benefits from the methods disclosed of the present disclosure. For example, in some embodiments, the patient may be a human male, a human female, or any other mammal.

The implementations discussed herein may include devices such as urological access devices or sheaths. For example, such devices can be configured to be placed on or within a body of a person or patient.

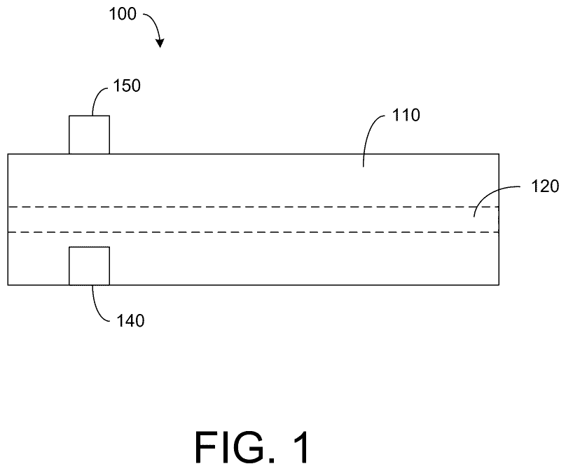

FIG. 1 schematically illustrates a device or apparatus 100 according to an implementation. The apparatus 100 includes an elongate member 110 that defines a lumen 120. The elongate member 110 may be a tubular member or cylindrical in shape and may be configured to be placed such that at least a portion of the elongate member 110 may be disposed within a body of a patient. For example, in some implementations, the elongate member 110 may be disposed within a body of a patient such as to provide access to a location within or internal to the body of the patient. In some implementations, a doctor or physician may access the internal location within the body of the patient via the lumen 120 defined by the elongate member 110. In some implementations, a secondary medical device, such as a scope, a lithotripsy device, or other medical device may be inserting through the lumen 120 of the elongate member 110 to or towards the internal location within the body of the patient.

In the illustrated implementation, the apparatus 100 also includes a flow meter 140. The flow meter 140 is configured to detect or measure fluid flow at a location proximate the apparatus 100. For example, in some implementations, the flow meter 140 is configured to detect or measure fluid flow within the lumen 120 defined by the elongate member 110. In some implementations, the flow meter 140 may be disposed within the lumen 120 defined by the elongate member 110.

In the illustrated implementation, the apparatus 100 includes a pressure sensor 150. The pressure sensor 150 is configured to detect or measure a pressure at a location proximate the apparatus 100. For example, in some implementations, the pressure sensor 150 is disposed at a distal end portion of the elongate member 110 and is configured to detect or measure a pressure within the body of the patient proximate the elongate member 110. For example, in some implementations, the pressure sensor 150 is configured to measure the pressure in a kidney or a bladder of a patient. In other implementations, the pressure sensor 150 is configured to measure the pressure within a different portion of the body of the patient. In some implementations, the pressure sensor 150 is configured to measure pressures between 0 and 400 mmHg.

In use, in some implementations, the apparatus 100 may be used in medical procedures. The elongate member 110 may be inserted into the body of the patient such that a proximal end portion of the elongate member 110 extends from the body of the patient and the distal end portion of the elongate member 110 is disposed within the body of the patient. A physician may then access the internal location within the body of the patient via the lumen 120 defined by the elongate member 110. The physician may utilize a secondary medical device by, for example, inserting such secondary medical device into the lumen 120.

In some implementations, the flow meter 140 is configured to detect or measure fluid flow within the lumen 120. For example, the fluid flow may be measured during the medial procedure and may be displayed to the physician on a display.

In some implementations, the pressure sensor 150 is configured to detect or measure a pressure near the elongate member 110. For example, the pressure may be measured during the medial procedure and may be displayed to the physician on a display.

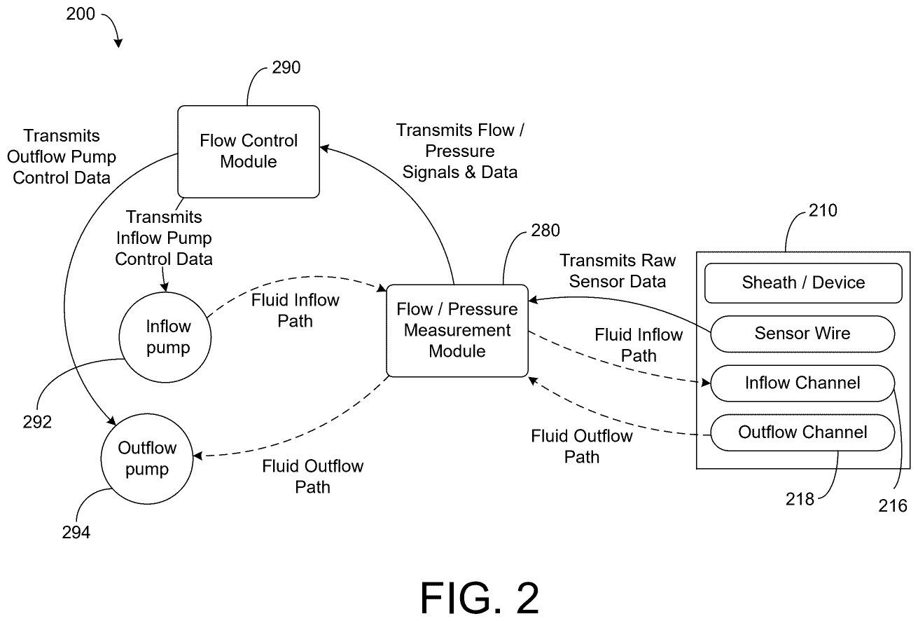

FIG. 2 schematically illustrates a system 200 according to an implementation. The system includes an elongate member 210 that defines various lumen or channels. In the illustrated implementation, the elongate member 210 includes or defines an inflow channel 216 and an outflow channel 218. The elongate member 210 may be a tubular member or cylindrical in shape and may be configured to be placed such that at least a portion of the elongate member 210 may be disposed within a body of a patient. For example, in some implementations, the elongate member 210 may be disposed within a body of a patient such as to provide access to a location within or internal to the body of the patient. In some implementations, a doctor or physician may access the internal location within the body of the patient via the lumen defined by the elongate member 210. In some implementations, a secondary medical device, such as a scope, a lithotripsy device, or other medical device may be inserting through the lumen of the elongate member 210 to or towards the internal location within the body of the patient.

In the illustrated implementation, the system 200 includes a flow/pressure measurement module 280. In some implementations, the flow/pressure measurement module 280 is configured to detect or measure fluid flow within lumens defined by the elongate member 210 and pressure proximate the elongate member 210. In the illustrated implementation, the flow/pressure measurement module 280 includes a flow meter. The flow meter is configured to detect or measure fluid flow at a location proximate the apparatus 200. For example, in some implementations, the flow meter is configured to detect or measure fluid flow within the lumen defined by the elongate member 210. In some implementations, the flow meter is configured to detect or measure fluid flow within the inflow channel 216, or the outflow channel 218, or both. In some implementations, the measurement module 280 is configured to filter raw data to reduce the noise within the data. In some implementations, the measurement module 280 includes an analog or digital components configured to help filter the raw data.

In the illustrated implementation, the system 200 includes a pressure sensor. The pressure sensor is configured to detect or measure a pressure at a location proximate the elongate member 210. For example, in some implementations, the pressure sensor is disposed at a distal end portion of the elongate member 210 and is configured to detect or measure a pressure within the body of the patient proximate the elongate member 210.

The flow/pressure measurement module 280 is operatively coupled to a flow control module 290. The flow control module 290 is configured to control the fluid flow within the inflow channel or lumen 216 and the outflow channel or lumen 218. In the illustrated implementation, the flow control module 290 is operatively coupled to an inflow pump 292. The inflow pump 292 is configured to pump fluid into the inflow channel 216. In the illustrated implementation, the flow control module 290 is also operatively coupled to an outflow pump 294. The outflow pump 294 is operatively coupled to the outflow channel 218 and is configured to pump fluid out of a body of a patient via the outflow channel 218. In some implementations, the flow control module 290 is configured to aspirate, irrigate, or limit the fluid flow. The flow control module 290 may include or be operatively coupled to a user interface that may include, for example, a touch screen. In some implementations, the flow control module 290 may also control the temperature or provide heated or warmed fluid.

In some implementations, the flow/pressure measurement module 280 and the flow control module 290 are incorporated together in a single unit. In other implementations, the flow/pressure measurement module 280 and the flow control module 290 are separate units or devices and are operatively coupled. For example, the flow/pressure measurement module 280 and the flow control module 290 may be operatively coupled together via a wire or other mechanism.

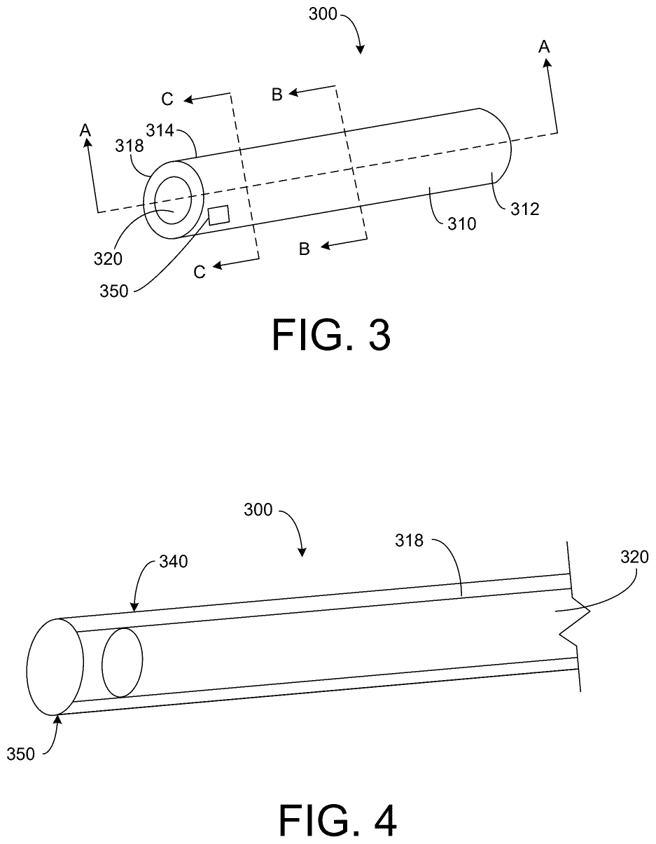

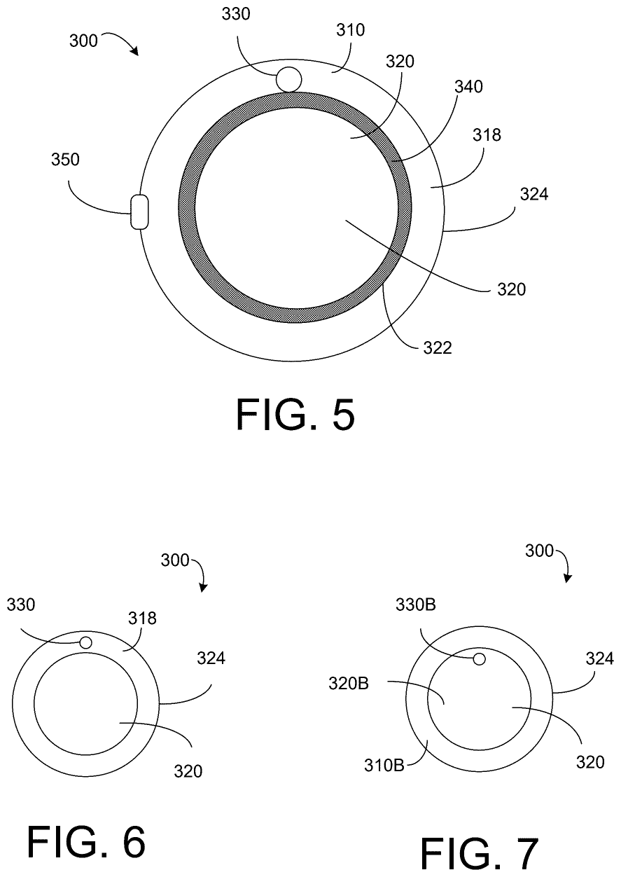

FIG. 3 is a perspective view of a device or apparatus 300 according to an implementation. FIG. 4 is a cross-sectional view of the device 300 taken along line A-A of FIG. 3. FIG. 5 is a cross-sectional view of the device 300 taken along line C-C of FIG. 3. FIG. 6 is a cross-sectional view of the device 300 taken along line B-B of FIG. 3.

The apparatus 300 includes an elongate member 310 that includes a proximal end portion 312 and a distal end portion 314. The elongate member 310 defines a lumen 320. In the illustrated implementation, the elongate member 310 has a sidewall 318 that defines the lumen 320. More specifically, the sidewall 318 has an inner surface 322 that defines the lumen 320 and an outer surface 324 opposite the inner surface 322.

The elongate member 310 may be a tubular member or cylindrical in shape and may be configured to be placed such that at least a portion of the elongate member 310 may be disposed within a body of a patient. For example, in some implementations, the elongate member 310 may be disposed within a body of a patient such as to provide access to a location within or internal to the body of the patient. In some implementations, a doctor or physician may access the internal location within the body of the patient via the lumen 320 defined by the elongate member 310. In some implementations, a secondary medical device, such as a scope, a lithotripsy device, or other medical device may be inserting through the lumen 320 of the elongate member 310 to or towards the internal location within the body of the patient.

In the illustrated implementation, the apparatus 300 also includes a flow meter 340. The flow meter 340 is configured to detect or measure fluid flow at a location proximate or close to the apparatus 300. In the illustrated implementation, the flow meter 340 may be disposed within the lumen 320 defined by the elongate member 310. In some implementations, the flow meter 340 is coupled to the inner surface 322 of the sidewall 318.

In the illustrated implementation, the apparatus 300 includes a pressure sensor 350. The pressure sensor 350 is configured to detect or measure a pressure at a location proximate or near the apparatus 300. For example, in the illustrated implementation, the pressure sensor 350 is disposed at the distal end portion 314 of the elongate member 310 and is configured to detect or measure a pressure within the body of the patient proximate or near the elongate member 310.

In the illustrated implementation, the apparatus 300 includes a wire 330. The wire 330 is operatively coupled to the flow meter 340 and the pressure sensor 350. The wire 330 is also operatively coupled to a display device or control modules that allow the data from the flow meter 340 and pressure sensor 350 to be displayed and/or analyzed. While only one wire is illustrated, in some implementations, the apparatus 300 includes a plurality of wires.

In the illustrated implementation, the wire 330 or at least a portion of the wire is disposed within the sidewall 318. For example, a portion of the wire 330 may be embedded within the material that forms the sidewall 318. As illustrated in FIG. 7, in other implementations, a wire 330B of the device may be disposed within the lumen 320B of the elongate member 310B.

In use, in some implementations, the apparatus 300 may be used in medical procedures. The elongate member 310 may be inserted into the body of the patient such that the proximal end portion 312 of the elongate member 310 extends from the body of the patient and the distal end portion 314 of the elongate member 310 is disposed within the body of the patient. A physician may then access the internal location within the body of the patient via the lumen 320 defined by the elongate member 310. The physician may utilize a secondary medical device by, for example, inserting such secondary medical device into the lumen 320.

In some implementations, the flow meter 340 is configured to detect or measure fluid flow within the lumen 320. For example, the fluid flow may be measured during the medial procedure and may be displayed to the physician on a display.

In some implementations, the pressure sensor 350 is configured to detect or measure a pressure near the elongate member 310. For example, the pressure may be measured during the medial procedure and may be displayed to the physician on a display.

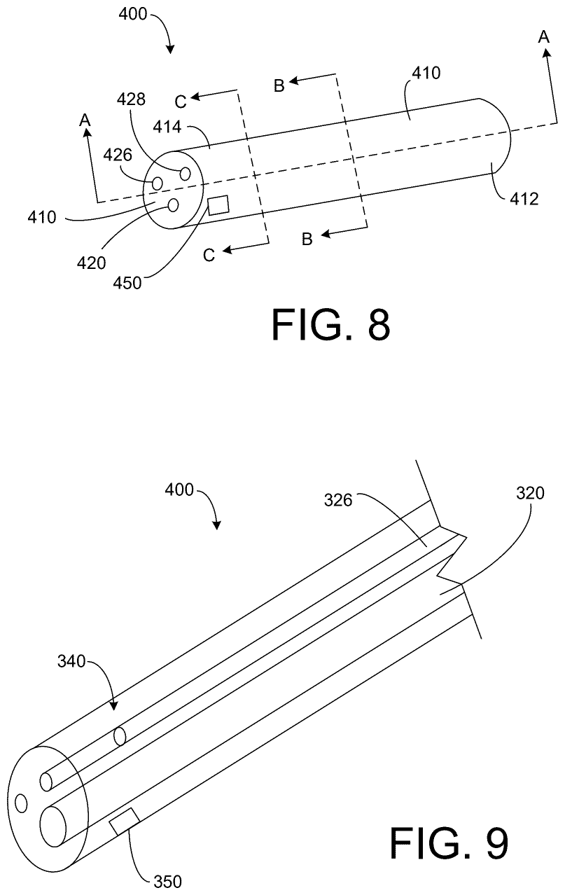

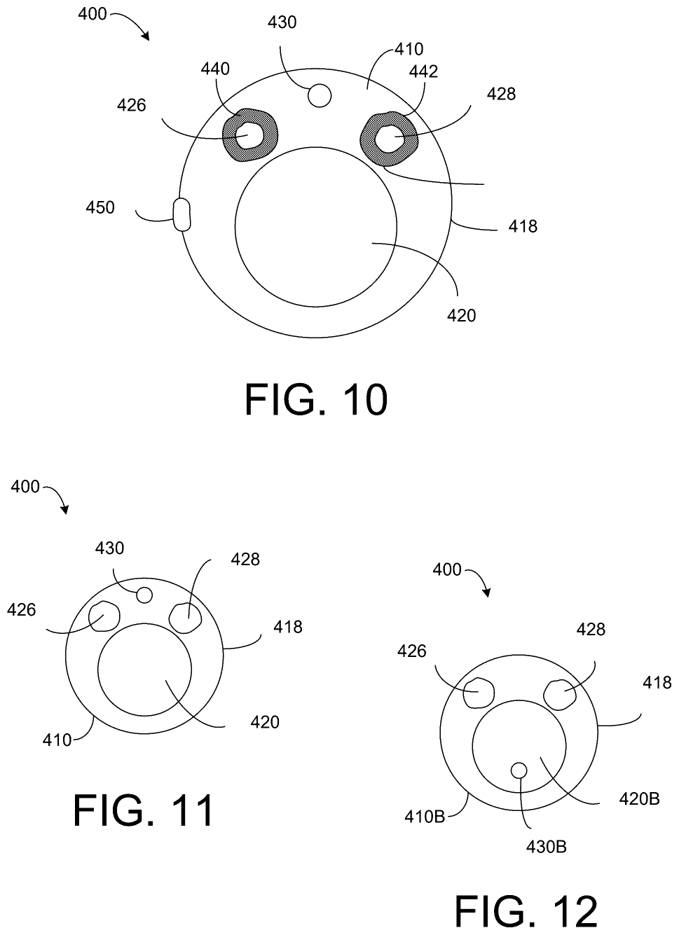

FIG. 8 is a perspective view of a device or apparatus 400 according to an implementation. FIG. 9 is a cross-sectional view of the device 400 taken along line A-A of FIG. 8. FIG. 10 is a cross-sectional view of the device 400 taken along line C-C of FIG. 8. FIG. 11 is a cross-sectional view of the device 400 taken along line B-B of FIG. 8.

The apparatus 400 includes an elongate member 410 that includes a proximal end portion 412 and a distal end portion 414. The elongate member 410 defines a first lumen 420, a second lumen 426, and a third lumen 428. The first lumen 420 may be a working channel. The second lumen 426 may be an inflow channel. The third lumen 428 may be an outflow channel. In the illustrated implementation, the elongate member 410 has a sidewall 418 that defines the first lumen 420, the second lumen 426, and the third lumen 428. More specifically, the sidewall 418 has an inner surface that define the lumens and an outer surface.

The elongate member 410 may be a tubular member or cylindrical in shape and may be configured to be placed such that at least a portion of the elongate member 410 may be disposed within a body of a patient. For example, in some implementations, the elongate member 410 may be disposed within a body of a patient such as to provide access to a location within or internal to the body of the patient. In some implementations, a doctor or physician may access the internal location within the body of the patient via the first lumen 420 defined by the elongate member 410. In some implementations, a secondary medical device, such as a scope, a lithotripsy device, or other medical device may be inserting through the first lumen 420 of the elongate member 410 to or towards the internal location within the body of the patient.

In the illustrated implementation, the apparatus 400 also includes a first flow meter 440 and a second flow meter 442. The flow meters 440 and 442 are configured to detect or measure fluid flow at a location proximate or close to the apparatus 400. In the illustrated implementation, the first flow meter 440 is disposed within the lumen 426 defined by the elongate member 410 and is configured to detect or measure fluid flow within lumen 426. In some implementations, the first flow meter 440 is coupled to an inner surface of the sidewall 418. In the illustrated implementation, the second flow meter 442 is disposed within the lumen 428 defined by the elongate member 410 and is configured to detect or measure fluid flow within lumen 428. In some implementations, the second flow meter 442 is coupled to an inner surface of the sidewall 418.

In the illustrated implementation, the apparatus 400 includes a pressure sensor 450. The pressure sensor 450 is configured to detect or measure a pressure at a location proximate or near the apparatus 400. For example, in the illustrated implementation, the pressure sensor 450 is disposed at the distal end portion 414 of the elongate member 410 and is configured to detect or measure a pressure within the body of the patient proximate or near the elongate member 410.

In the illustrated implementation, the apparatus 400 includes a wire 430. The wire 430 is operatively coupled to the flow meters 440 and 442 and the pressure sensor 450. The wire 430 is also operatively coupled to a display device or control modules that allow the data from the flow meters 440 and 442 and pressure sensor 450 to be displayed and/or analyzed.

In the illustrated implementation, the wire 430 or at least a portion of the wire is disposed within the sidewall 418. For example, a portion of the wire 430 may be embedded within the material that forms the sidewall 418. As illustrated in FIG. 12, in other implementations, a wire 430B of the device may be disposed within the lumen 420B of the elongate member 410B.

In use, in some implementations, the apparatus 400 may be used in medical procedures. The elongate member 410 may be inserted into the body of the patient such that the proximal end portion 412 of the elongate member 410 extends from the body of the patient and the distal end portion 414 of the elongate member 410 is disposed within the body of the patient. A physician may then access the internal location within the body of the patient via the lumen 420 defined by the elongate member 410. The physician may utilize a secondary medical device by, for example, inserting such secondary medical device into the lumen 420.

In some implementations, the first flow meter 440 is configured to detect or measure fluid flow within the second lumen 426 such as an inflow lumen. For example, the fluid flow may be measured during the medial procedure and may be displayed to the physician on a display. In some implementations, the second flow meter 442 is configured to detect or measure fluid flow within the third lumen 428 such as an outflow lumen. For example, the fluid flow may be measured during the medial procedure and may be displayed to the physician on a display.

In some implementations, the pressure sensor 450 is configured to detect or measure a pressure near the elongate member 410. For example, the pressure may be measured during the medial procedure and may be displayed to the physician on a display.

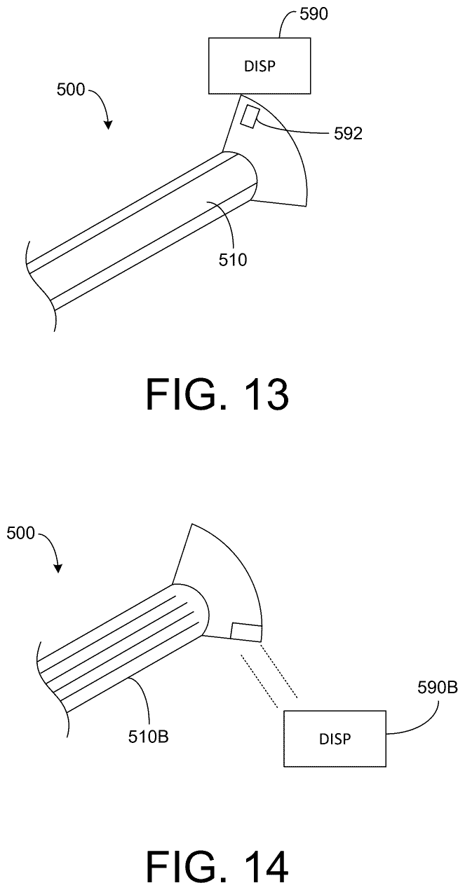

FIG. 13 is a perspective view of a device 500 according to an implementation. Similar to the devices 100, 200, 300, and 400 the device 500 includes an elongate member 510 and flow meters (not illustrated) and sensors (not illustrated). The device 500 includes a display 590. The display 590 is operatively coupled to the flow meters and sensors and is configured to provide a display, such as a visual display, to a physician. For example, in some implementations, the display 590 is configured to display the flow within the device or the pressure within the body of the patient as detected or measured by the flow meters and the sensors.

As illustrated in FIG. 13, the display 590 may be coupled to an end portion of the elongate member 510. In some implementations, the display 590 is removably coupled to the elongate member 510. For example, the display 590 may be removably coupled to the elongate member 510 via a clip 592 or other mechanical device. In other implementations, the display 590 is fixedly coupled to the elongate member 510.

As best illustrated in FIG. 14, in some implementations, the display 590B is wirelessly coupled to the elongate member 510B. Accordingly, the display 590B may be disposed apart or spaced from the elongate member 510B. In some implementations, the display 590B is coupled to or communicates with the elongate member 510B via Bluetooth or other wireless system.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
an elongate member defining a lumen, the elongate member being configured to be disposed within a body of a patient;
a flow meter configured to measure fluid flow through the lumen of the elongate member; and
a pressure sensor.

2. The apparatus of claim 1, wherein the flow meter is disposed within the lumen defined by the elongate member.

3. The apparatus of claim 1, wherein the pressure sensor is disposed outside of the lumen defined by the elongate member.

4. The apparatus of claim 1, wherein the elongate member includes a sidewall that has an inner surface that defines the lumen and an outer surface opposite the inner surface, the flow meter is disposed adjacent the inner surface of the sidewall.

5. The apparatus of claim 1, wherein the elongate member includes a sidewall that has an inner surface that defines the lumen and an outer surface opposite the inner surface, the pressure sensor is disposed adjacent the outer surface of the sidewall.

6. The apparatus of claim 1, wherein the lumen defined by the elongate member is a first lumen, the elongate member defining a second lumen.

7. The apparatus of claim 1, wherein the lumen defined by the elongate member is a first lumen, the elongate member defining a second lumen and a third lumen.

8. The apparatus of claim 1, wherein the lumen defined by the elongate member is a first lumen, the elongate member defining a second lumen and a third lumen, the first lumen being configured to receive a secondary medical device, the first lumen configured to be an inflow channel, the second lumen configured to be an outflow channel.

9. The apparatus of claim 1, wherein the lumen defined by the elongate member is a first lumen, the flow meter being a first flow meter, the elongate member defining a second lumen, the apparatus further comprising:
a second flow meter disposed within the second lumen.

10. The apparatus of claim 1, wherein the lumen defined by the elongate member is a first lumen, the flow meter being a first flow meter, the elongate member defining a second lumen and a third lumen, the apparatus further comprising:
a second flow meter disposed within the second lumen.

11. The apparatus of claim 1, further comprising:
a wire operatively coupled to the flow meter, at least a portion of the wire being disposed within lumen defined by the elongate member.

12. The apparatus of claim 1, further comprising:
a wire operatively coupled to the flow meter, at least a portion of the wire being disposed within a sidewall of the elongate member.

13. The apparatus of claim 1, further comprising:
a display member coupled to a proximal end portion of the elongate member.

14. The apparatus of claim 1, further comprising:
a display member configured to be disposed a distance from the elongate member and configured to receive information regarding the fluid flow within the lumen of the elongate member.

15. The apparatus of claim 1, further comprising:
a display member configured to be disposed a distance from the elongate member and configured to wirelessly communicate with the elongate member.

16. An apparatus, comprising:
an elongate member defining a first lumen, a second lumen and a third lumen, the elongate member being configured to be disposed within a body of a patient, the first lumen being configured to receive a secondary medical device;
a first flow meter configured to measure fluid flow through the second lumen of the elongate member; and
a second flow meter configured to measure fluid flow through the third lumen of the elongate member.

17. The apparatus of claim 16, further comprising:
a pressure sensor.

18. The apparatus of claim 16, further comprising:
a pressure sensor coupled to an end portion of the elongate member.

19. The apparatus of claim 16, further comprising:
a display member coupled to the elongate member.

20. The apparatus of claim 16, further comprising:
a display member configured to be disposed a distance from the elongate member and configured to wirelessly communicate with the elongate member.
